SEQUENCE LISTING (6 sequences)

SEQ ID NO: 1 - DNA, 22 bp, synthetic construct
  source 1..22: mol_type = other DNA
ataaactgacctggcggcggtg

SEQ ID NO: 2 - DNA, 25 bp, synthetic construct
  source 1..25: mol_type = other DNA
cctcatcctcttctgaagaacctaa

SEQ ID NO: 3 - DNA, 31 bp, Homo sapiens
  source 1..31: mol_type = genomic DNA
atgagccaccgccgccaggtcagtttatttt

SEQ ID NO: 4 - DNA, 61 bp, Homo sapiens
  source 1..61: mol_type = genomic DNA
ctctgattttaggttcttcagaagaggatgaggtcattcaaccaggtttcatcagccccag

SEQ ID NO: 5 - DNA, 22 bp, synthetic construct
  source 1..22: mol_type = other DNA
gaggtggtggaactacctagcc

SEQ ID NO: 6 - DNA, 25 bp, synthetic construct
  source 1..25: mol_type = other DNA
ataatccaggcctaactatcctctc

(19) United States
(12) Patent Application Publication
(10) Pub. No.: US 2024/0150764 A1
(43) Pub. Date: May 9, 2024

(54) THERAPEUTIC SPLICE-SWITCHING OLIGONUCLEOTIDES FOR CANCER

(71) Applicants: Noah Basri, St. Louis, MO (US); Ting Wang, St. Louis, MO (US)

(72) Inventors: Noah Basri, St. Louis, MO (US); Ting Wang, St. Louis, MO (US)

(73) Assignee: Washington University, St. Louis, MO (US)

(21) Appl. No.: 18/503,495

(22) Filed: Nov. 7, 2023

Related U.S. Application Data
(60) Provisional application No. 63/423,289, filed on Nov. 7, 2022.

(51) Int. Cl. C12N 15/113

(52) U.S. Cl. CPC C12N 15/1135; C12N 2310/11; C12N 2310/315; C12N 2310/321; C12N 2320/33

(57) ABSTRACT

The present disclosure provides for splice-switching oligonucleotides (SSOs) targeted to a nucleic acid encoding a transposable element (TE)-driven isoform of LIN28B, such as AluJb-LIN28B. The SSOs may be targeted to an exon-intron or intron-exon junction to inhibit splicing and expression of the TE-driven isoform of LIN28B. Methods of treating cancer, particularly cancers expressing AluJb-LIN28B, comprising administering the SSOs are also provided.

### CROSS-REFERENCE TO RELATED APPLICATIONS

This application claims priority from U.S. Provisional Application Ser. No. 63/423,289 filed on 7 Nov. 2022, which is incorporated herein by reference in its entirety.

### STATEMENT REGARDING FEDERALLY SPONSORED RESEARCH OR DEVELOPMENT

This invention was made with government support under CA091842, HG007175, ES026699, and HG009391 awarded by the National Institutes of Health. The government has certain rights in the invention.

### MATERIAL INCORPORATED-BY-REFERENCE

The Sequence Listing, which is a part of the present disclosure, includes a computer-readable form comprising nucleotide and/or amino acid sequences of the present invention (file name 020346-US-NP_Sequence_Listing.xml created on 3 Nov. 2023; 6,416 bytes). The subject matter of the Sequence Listing is incorporated herein by reference in its entirety.

### FIELD

The present disclosure generally relates to splice-switching oligonucleotides (SSOs) targeted to a transposable element (TE)-driven isoform of LIN28B and methods of use thereof.

### SUMMARY

Among the various aspects of the present disclosure, a method of treating cancer in a subject in need thereof is provided. The method comprises administering to the subject a splice-switching oligonucleotide (SSO) targeted to a nucleic acid encoding a transposable element (TE)-driven isoform of LIN28B.

In some embodiments, the SSO is targeted to an exon-intron junction or an intron-exon junction of the TE-driven isoform of LIN28B. In some embodiments, the TE-driven isoform of LIN28B is AluJb-LIN28B.

In some embodiments, the SSO is at least 50% identical to SEQ ID NO: 1 or SEQ ID NO: 2, or to a corresponding reverse, complement, or reverse-complement sequence thereof. In other embodiments, the SSO is at least 50% identical to SEQ ID NO: 3 or SEQ ID NO: 4, or to a corresponding reverse, complement, or reverse-complement sequence thereof and is at least 10 nucleotides in length. In yet other embodiments, the SSO comprises SEQ ID NO: 1, SEQ ID NO: 2, or a corresponding reverse, complement, or reverse-complement sequence thereof.

In some embodiments, the subject has a cancer that expresses AluJb-LIN28B, including cancer selected from liver cancer, bladder cancer, urothelial cancer, breast cancer, lung cancer, ovarian cancer, melanoma, and stomach cancer.

In some embodiments, the SSO comprises at least one chemical modification, including chemical modification(s) selected from the group consisting of 2′-O-methoxyethyl modification, phosphorothioate internucleotide linkage, and combinations thereof.

In some embodiments, the nucleic acid encoding the TE-driven isoform of LIN28B is a pre-mRNA. In some embodiments, administering the SSO reduces at least one of a mRNA level of the TE-driven isoform of LIN28B, a protein level of the TE-driven isoform of LIN28B, and cancer cell viability in the subject.

Among further aspects of the present disclosure, a pharmaceutical composition is provided. The pharmaceutical composition comprises a splice-switching oligonucleotide (SSO) targeted to a nucleic acid encoding a transposable-element (TE)-driven isoform of LIN28B and a pharmaceutically acceptable carrier.

In some embodiments, the SSO is targeted to an exon-intron junction of the TE-driven isoform of LIN28B. In some embodiments, the TE-driven isoform of LIN28B is AluJb-LIN28B.

In some embodiments, the SSO is at least 50% identical to SEQ ID NO: 1 or SEQ ID NO: 2, or to a corresponding reverse, complement, or reverse-complement sequence thereof. In other embodiments, the SSO is at least 50% identical to SEQ ID NO: 3 or SEQ ID NO: 4, or to a corresponding, reverse, complement, or reverse-complement sequence thereof, and is at least 10 nucleotides in length. In yet other embodiments, the SSO comprises SEQ ID NO: 1, SEQ ID NO: 2, or a corresponding reverse, complement, or reverse-complement sequence thereof.

In some embodiments, the SSO comprises at least one chemical modification, including chemical modification(s) selected from the group consisting of 2′-O-methoxyethyl modification, phosphorothioate internucleotide linkage, and combinations thereof.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1 is a pie chart showing transposable elements constitute significant proportions of genome content in accordance with the present disclosure.

FIG. 2 is a schematic showing intron 1 retention via SSO1 or exon 2 skipping via SSO2 in accordance with the present disclosure.

FIG. 3 is a schematic showing the experimental plan in accordance with the present disclosure.

FIG. 4 includes images and a line graph showing SSO1 potently reduces H1299 viability in accordance with the present disclosure. Cells were treated with SSO1 (new sso), scrambled control (new scr), or puromycin at increasing concentrations for 5 days post-transfection and viability (OD450 absorbance) was measured. The EC₅₀ of SSO1 was 20.77 nM.

FIG. 5 is a line graph showing SSO2 potently reduces H1299 viability in accordance with the present disclosure. Cells were treated with SSO2 (sso), scrambled control (scr), or puromycin at increasing concentrations and viability (OD450 absorbance) was measured at 5 days post-transfection. The EC₅₀ SSO1 was 21.86 nM.

FIG. 6 is a line graph showing SSO1 significantly reduced H1299 viability 3 days post-transfection in accordance with the present disclosure. Cells were treated with either 40 nm SSO1 (sso1), 40 nm scrambled control (scr1), or puromycin (puro) and viability (OD450 absorbance) was measured starting at day 0 post-transfection. Viability of untreated cells (0) is also shown.

FIG. 7 is a line graph showing SSO2 significantly reduced H1299 viability 5 days post-transfection in accordance with the present disclosure. Cells were treated with 40 nm SSO2 (sso), 40 nm scrambled control (scr), or puromycin (puro) and viability (OD450 absorbance) was measured starting at day 0 post-transfection. Untreated cells (unTx) are also shown.

FIG. 8 is a line graph showing that SSO1 does not reduce viability of HEK293 cells in accordance with the present disclosure. HEK293 cells were treated with 40 nm SSO1 (sso 40), scrambled control (scr 40), or puromycin and viability (OD450 absorbance) was measured starting at day 0 post-transfection. Untreated cells (untx) are also shown.

FIG. 9A-FIG. 9B include graphics showing that RNA-seq confirmed LIN28B knockdown in accordance with the present disclosure. FIG. 9A shows LIN28B expression from cells treated with 40 nm scrambled control (40-SCR-CHX-RNA), 40 nm SSO1 (40-SSO-CHX-RNA), or untreated cells (WT-untreated). Expression of HMGA2, a let-7 regulated oncogene, is shown in FIG. 9B.

FIG. 10 includes images showing SSO treatment reduces H1299 colony formation ability in accordance with the present disclosure. Cells were treated for 2 days with SSO1, scrambled control 1 (SCR1), SSO1, or scrambled control 2 (SCR2) prior to sparse seeding.

FIG. 11 includes Western blot images showing SSO1 reduces in-frame LIN28B protein in H1299 in accordance with the present disclosure. HEK and H1299 cells were treated with either 40 or 60 nM SSO or equal concentration of scrambled control (SCR). Western blot for LIN28B protein expression was performed 7 days after transfection. HEK293-derived canonical LIN28B is predicted to be ˜3 kDa smaller than AluJb-LIN28B.

FIG. 12 is a schematic showing generation of H1299-Luc cells in accordance with the present disclosure.

### DETAILED DESCRIPTION

The present disclosure is based, at least in part, on the discovery that a specific splice-switching oligonucleotide (SSO) can be directed against a transposable-element (TE)-driven, cancer-specific isoform of LIN28B. As shown herein, administration of an AluJb-LIN28B-targeted SSO inhibits LIN28B mRNA and protein expression in cancer cells and reduces cancer cell viability with high potency without affecting normal cells (see e.g., Example 1).

Transposable elements are remnants of ancient viruses and contribute cis-regulatory elements genome-wide. Normally TE-derived promoters are repressed with DNA methylation; however, in cancer, global hypomethylation results in activation of cryptic TE-derived promoters with downstream gene expression.

The AluJb TE is located ˜20 kb upstream of the canonical LIN28B promoter and drives a tumor-specific isoform of LIN28B in several cancer types. The AluJb-derived isoform constitutes >90% of LIN28B expression in cancers where this tumor-specific isoform is expressed.

Primary liver cancer is the seventh-most frequently occurring cancer in the world and the second-most common cause of cancer mortality. Of the 10-15% of all hepatocellular carcinoma (HCC) tumors in TCGA that express LIN28B, the tumor-specific TE-derived isoform AluJb-LIN28B is expressed in 91.5% of them, accounting for greater than 90% of total LIN28B expression in those tumors. Post-resection analysis of HCC from a Taiwanese cohort over a 17 year period revealed LIN28B expression in 37% of cases.

More than half of all HCC cases worldwide are diagnosed in China, where it is the 4th most commonly diagnosed cancer, and the 2nd most common cause of cancer-associated mortality. Compared to HCC diagnosed in Europe and North America, HCC diagnosed in China has its own etiological, demographic, biological and clinical nuances, which may explain the 37% LIN28B positivity rate in the Taiwanese HCC cohort. Additionally, HCC is among the fastest rising causes of cancer-related deaths in the US. The TE-derived LIN28B isoform abrogated by the SSOs described herein is also expressed in numerous other cancers accounting for significant contributions to LIN28B expression. The SSOs described herein would be a first-in-class treatment as, currently, there are believed to be no therapies in clinical trials directed against the cancer-specific isoform of LIN28B.

Splice-switching oligonucleotides (SSOs) are short, synthetic nucleic acid sequences designed to bind a target pre-mRNA through antisense Watson-Crick base-pairing, thereby disrupting canonical splicing programs and associated gene expression. Steric hindrance of spliceosome function alters the mRNA reading frame, introducing a premature termination codon with subsequent gene product degradation through nonsense-mediated mRNA decay.

Described herein is the application of this biological rationale to generate a fully 2′-O-methoxyethyl modified SSO containing phosphorothioate internucleotide linkages, directed against the transposable-element (TE)-driven, cancer-specific isoform of LIN28B, a well-characterized and historically “undruggable” oncogene. LIN28B represses let-7 microRNAs, which pleiotropically suppress numerous oncogenes like MYC, KRAS, HMGA2, the immune checkpoint PD-L1, and others through cell intrinsic RNAi. Accordingly, the presence of this tumor-specific isoform portends a significantly poorer prognosis in the ˜10-15% of hepatocellular carcinoma (HCC) that express it. Beyond HCC, the TE-derived LIN28B isoform targeted by the SSO sporadically contributes to LIN28B gene expression in other cancers including melanoma, lung squamous cell carcinoma, cervical cancer, uterine cancer, head and neck squamous cell carcinoma, bladder cancer and stomach cancer.

As shown herein, TE-directed LIN28B-abrogating SSOs demonstrated selective viability reductions in cells expressing AluJb-LIN28B, but not cells expressing the canonical LIN28B isoform (see e.g., Example 1).

While antisense oligonucleotides targeting the canonical LIN28B transcript have previously been described in the art, the present disclosure is believed to be the first to describe SSOs targeted to a tumor-specific, TE-driven isoform of LIN28B.

### Splice-Switching Oligonucleotide (SSO)

The present disclosure provides for a splice-switching oligonucleotide (SSO) targeted to a nucleic acid encoding a transposable-element (TE)-driven isoform of LIN28B.

As described herein, splice-switching oligonucleotides (SSOs) can be directed against oncogenes or cancer-specific isoforms to treat cancer and reduce cancer cell viability without affecting normal cells. Generally, a SSO in accordance with the present disclosure is a short (e.g., about 10 to about 30 nucleotides in length), synthetic, antisense oligonucleotide designed to specifically modulate splicing of a target gene. SSOs target specific sequences on a pre-mRNA transcript through antisense Watson-Crick base pairing. The SSO disrupts canonical splicing programs, such as through steric hindrance of spliceosome function, resulting in alteration of the reading frame and knockdown of the associated gene product. SSOs can be designed to disrupt splicing through various mechanisms. For example, as shown herein, SSOs may be designed to target junctions between introns and exons on a pre-mRNA transcript and result in the retention of an intron or exon skipping.

In some embodiments, the SSO in accordance with the present disclosure comprises at least one chemical modification. SSOs may be chemically modified to modulate various properties, such as stability, nuclease resistance, affinity, pharmacodynamic properties, or therapeutic efficacy. Chemical modifications for SSOs and other antisense oligonucleotides are known in the art and may include for example modifications to the backbone, sugar moiety, or base moiety of a nucleotide. Non-limiting examples of suitable chemical modifications include a phosphorothioate internucleotide linkage, 2′-O-methoxyethyl modification, 2′-O-methyl modification, locked nucleic acid, or phosphorodiamidate morpholinos. In some preferred embodiments, the chemical modification is a phosphorothioate internucleotide linkage, 2′-O-methoxyethyl modification, or combination thereof. In some embodiments, the SSO is “fully modified”. As used herein, a fully modified SSO comprises a chemical modification at every nucleotide and/or linkage comprised in the SSO.

### Transposable Element-Driven Isoform of LIN28B

Generally, alternative transcription start sites contributed by transposable elements (TEs) throughout the genome can become activated in cancer, resulting in TE-driven isoforms of canonical genes. TE-driven expression of the oncogene LIN28B has been identified in various cancers and has been associated with worse outcomes. As described herein, SSOs targeted to a TE-driven isoform of LIN28B can be used to reduce cancer cell viability without adversely affecting normal cells.

In some embodiments, the TE-driven isoform of LIN28B is AluJb-LIN28B. The AluJb TE is located ˜20 kb upstream of the canonical LIN28B promoter and drives a tumor-specific isoform of LIN28B in several cancer types. The AluJb-derived isoform constitutes >90% of LIN28B expression in cancers where this tumor-specific isoform is expressed. As shown herein, SSOs targeted to AluJb-LIN28B demonstrated selective viability reductions in cells expressing AluJb-LIN28B (e.g., cancer cells), but not cells expressing the canonical LIN28B isoform (see e.g., Example 1).

In some embodiments, the SSO is targeted to an exon-intron junction of AluJb-LIN28B to disrupt splicing and LIN28B expression. For example, the SSO may be targeted or designed to hybridize to a sequence comprising a junction between exon 1 and intron 1 of AluJb-LIN28B. As such, the SSO can be derived from or complementary to the sequence

(SEQ ID NO: 3)
ATGAGCCACCGCCGCCAGGTCAGTTTATTTT

SEQ ID NO: 3 comprises the exon 1-intron 1 junction of AluJb-LIN28B, with the exon depicted in bold text and the intron depicted in italic text. As an example, the SSO can comprise the sequence ATAAACTGACCTGGCGGCGGTG (SEQ ID NO: 1), or a corresponding, reverse, complement, or reverse-complement thereof. As described herein, an SSO comprising SEQ ID NO: 1 (SSO1) was designed to hybridize to the underlined portion of SEQ ID NO: 3 and specifically and potently reduced cancer cell viability (see e.g., Example 1).

As another example, the SSO may be targeted or designed to hybridize to a sequence comprising a junction between intron 1 and exon 2 of AluJb-LIN28B. As such, the SSO can be derived from or complementary to the sequence:

(SEQ ID NO: 4)
CTCTGATTTTAGGTTCTTCAGAAGAGGATGAGG
TCATTCAACCAGGTTTCATCAGCCCCAG

SEQ ID NO: 4 comprises the intron 1-exon 2 junction of AluJb-LIN28B, with the exon depicted in in bold text and the intron depicted in italic text. As an example, the SSO can comprise the sequence CCTCATCCTCTTCTGAAGAACCTAA (SEQ ID NO: 2), or a corresponding, reverse, complement, or reverse-complement thereof. As described herein, an SSO comprising SEQ ID NO: 2 (SSO2) was designed to hybridize to the underlined portion of SEQ ID NO: 4 and specifically and potently reduced cancer cell viability (see e.g., Example 1).

SSOs that are variants of SEQ ID NO: 1 or SEQ ID NO: 2 which retain the function of targeting AluJb-LIN28B and consequently reducing expression of AluJb-LIN28B may also be used in the compositions and methods of the present disclosure. The making and use of such variants is within the skill of one in the art. For example, a tiling assay could be used to systematically vary the sequences of SEQ ID NO: 1 or SEQ ID NO: 2 and identify variants having the ability to hybridize with target AluJb-LIN28B sequences.

In one embodiment, such a variant SSO is at least 50% identical to SEQ ID NO: 1 or SEQ ID NO: 2 or a corresponding, reverse, complement, or reverse-complement thereof. In another embodiment, such a variant SSO is at least 66% identical to SEQ ID NO: 1 or SEQ ID NO: 2 or a corresponding, reverse, complement, or reverse-complement thereof. In another embodiment, such a variant SSO is at least 75% identical to SEQ ID NO: 1 or SEQ ID NO: 2 or a corresponding, reverse, complement, or reverse-complement thereof. In another embodiment, such a variant SSO is at least 80% identical to SEQ ID NO: 1 or SEQ ID NO: 2 or a corresponding, reverse, complement, or reverse-complement thereof. In another embodiment, such a variant SSO is at least 85% identical to SEQ ID NO: 1 or SEQ ID NO: 2 or a corresponding, reverse, complement, or reverse-complement thereof. In another embodiment, such a variant SSO is at least 90% identical to SEQ ID NO: 1 or SEQ ID NO: 2 or a corresponding, reverse, complement, or reverse-complement thereof. In another embodiment, such a variant SSO is at least 95% identical to SEQ ID NO: 1 or SEQ ID NO: 2 or a corresponding, reverse, complement, or reverse-complement thereof.

### Molecular Engineering

The following definitions and methods are provided to better define the present invention and to guide those of ordinary skill in the art in the practice of the present invention. Unless otherwise noted, terms are to be understood according to conventional usage by those of ordinary skill in the relevant art.

The term “transfection,” as used herein, refers to the process of introducing nucleic acids into cells by non-viral methods. The term “transduction,” as used herein, refers to the process whereby foreign DNA is introduced into another cell via a viral vector.

The terms “heterologous DNA sequence”, “exogenous DNA segment”, or “heterologous nucleic acid”, “transgene”, “exogenous polynucleotide” as used herein, each refers to a sequence that originates from a source foreign (e.g., non-native) to the particular host cell or, if from the same source, is modified from its original form. Thus, a heterologous gene in a host cell includes a gene that is endogenous to the particular host cell but has been modified through, for example, the use of DNA shuffling or cloning. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Sequences described herein can also be the reverse, the complement, or the reverse complement of the nucleotide sequences described herein. The RNA goes in the reverse direction compared to the DNA, but its base pairs still match (e.g., G to C). The reverse complementary RNA for a positive strand DNA sequence will be identical to the corresponding negative strand DNA sequence. Reverse complement converts a DNA sequence into its reverse, complement, or reverse-complement counterpart.

| Base | Name | Bases Represented | Complementary Base |
|---|---|---|---|
| A | Adenine | A | T |
| T | Thymidine | T | A |
| U | Uridine(RNA only) | U | A |
| G | Guanidine | G | C |
| C | Cytidine | C | G |
| Y | pYrimidine | C T | R |
| R | puRine | A G | Y |
| S | Strong(3Hbonds) | G C | S* |
| W | Weak(2Hbonds) | A T | W* |
| K | Keto | T/U G | M |
| M | aMino | A C | K |
| B | not A | C G T | V |
| D | not C | A G T | H |
| H | not G | A C T | D |
| V | not T/U | A C G | B |
| N | Unknown | A C G T | N |

Complementarity is a property shared between two nucleic acid sequences (e.g., RNA, DNA), such that when they are aligned antiparallel to each other, the nucleotide bases at each position will be complementary. Two bases are complementary if they form Watson-Crick base pairs.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

An “expression vector”, otherwise known as an “expression construct”, is generally a plasmid or virus designed for gene expression in cells. The vector is used to introduce a specific gene into a target cell, and can commandeer the cell's mechanism for protein synthesis to produce the protein encoded by the gene. Expression vectors are the basic tools in biotechnology for the production of proteins. The vector is engineered to contain regulatory sequences that act as enhancer and/or promoter regions and lead to efficient transcription of the gene carried on the expression vector. The goal of a well-designed expression vector is the efficient production of protein, and this may be achieved by the production of significant amount of stable messenger RNA, which can then be translated into protein. The expression of a protein may be tightly controlled, and the protein is only produced in significant quantity when necessary through the use of an inducer, in some systems however the protein may be expressed constitutively. As described herein, Escherichia coli is used as the host for protein production, but other cell types may also be used.

In molecular biology, an “inducer” is a molecule that regulates gene expression. An inducer can function in two ways, such as:
(i) By disabling repressors. The gene is expressed because an inducer binds to the repressor. The binding of the inducer to the repressor prevents the repressor from binding to the operator. RNA polymerase can then begin to transcribe operon genes. An operon is a cluster of genes that are transcribed together to give a single messenger RNA (mRNA) molecule, which therefore encodes multiple proteins.
(ii) By binding to activators. Activators generally bind poorly to activator DNA sequences unless an inducer is present. An activator binds to an inducer and the complex binds to the activation sequence and activates target gene. Removing the inducer stops transcription. Because a small inducer molecule is required, the increased expression of the target gene is called induction.

Repressor proteins bind to the DNA strand and prevent RNA polymerase from being able to attach to the DNA and synthesize mRNA. Inducers bind to repressors, causing them to change shape and preventing them from binding to DNA. Therefore, they allow transcription, and thus gene expression, to take place.

For a gene to be expressed, its DNA sequence (or polynucleotide sequence) must be copied (in a process known as transcription) to make a smaller, mobile molecule called messenger RNA (mRNA), which carries the instructions for making a protein to the site where the protein is manufactured (in a process known as translation). Many different types of proteins can affect the level of gene expression by promoting or preventing transcription. In prokaryotes (such as bacteria), these proteins often act on a portion of DNA known as the operator at the beginning of the gene. The promoter is where RNA polymerase, the enzyme that copies the genetic sequence and synthesizes the mRNA, attaches to the DNA strand.

Some genes are modulated by activators, which have the opposite effect on gene expression as repressors. Inducers can also bind to activator proteins, allowing them to bind to the operator DNA where they promote RNA transcription. Ligands that bind to deactivate activator proteins are not, in the technical sense, classified as inducers, since they have the effect of preventing transcription.

A “promoter” is generally understood as a nucleic acid control sequence that directs transcription of a nucleic acid. An inducible promoter is generally understood as a promoter that mediates transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

A “ribosome binding site”, or “ribosomal binding site (RBS)”, refers to a sequence of nucleotides upstream of the start codon of an mRNA transcript that is responsible for the recruitment of a ribosome during the initiation of translation. Generally, RBS refers to bacterial sequences, although internal ribosome entry sites (IRES) have been described in mRNAs of eukaryotic cells or viruses that infect eukaryotes. Ribosome recruitment in eukaryotes is generally mediated by the 5′ cap present on eukaryotic mRNAs.

A ribosomal skipping sequence (e.g., 2A sequence such as furin-GSG-T2A) can be used in a construct to prevent covalently linking translated amino acid sequences.

A “transcribable nucleic acid molecule” as used herein refers to any nucleic acid molecule capable of being transcribed into an RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position +1. With respect to this site all other sequences of the gene and its controlling regions can be numbered. Downstream sequences (i.e., further protein encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A construct of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal, or a plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using self-replicating primers, paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

Design, generation, and testing of the variant nucleotides, and their encoded polypeptides, having the above-required percent identities and retaining a required activity of the expressed protein is within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2, or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A. For example, the percent identity can be at least 80% or about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

Substitution refers to the replacement of one amino acid with another amino acid in a protein or the replacement of one nucleotide with another in DNA or RNA. Insertion refers to the insertion of one or more amino acids in a protein or the insertion of one or more nucleotides with another in DNA or RNA. Deletion refers to the deletion of one or more amino acids in a protein or the deletion of one or more nucleotides with another in DNA or RNA. Generally, substitutions, insertions, or deletions can be made at any position so long as the required activity is retained.

“Point mutation” refers to when a single base pair is altered. A point mutation or substitution is a genetic mutation where a single nucleotide base is changed, inserted, or deleted from a DNA or RNA sequence of an organism's genome. Point mutations have a variety of effects on the downstream protein product—consequences that are moderately predictable based upon the specifics of the mutation. These consequences can range from no effect (e.g., synonymous mutations) to deleterious effects (e.g., frameshift mutations), with regard to protein production, composition, and function. Point mutations can have one of three effects. First, the base substitution can be a silent mutation where the altered codon corresponds to the same amino acid. Second, the base substitution can be a missense mutation where the altered codon corresponds to a different amino acid. Or third, the base substitution can be a nonsense mutation where the altered codon corresponds to a stop signal. Silent mutations result in a new codon (a triplet nucleotide sequence in RNA) that codes for the same amino acid as the wild type codon in that position. In some silent mutations the codon codes for a different amino acid that happens to have the same properties as the amino acid produced by the wild type codon. Missense mutations involve substitutions that result in functionally different amino acids; these can lead to alteration or loss of protein function. Nonsense mutations, which are a severe type of base substitution, result in a stop codon in a position where there was not one before, which causes the premature termination of protein synthesis and can result in a complete loss of function in the finished protein.

Generally, conservative substitutions can be made at any position so long as the required activity is retained. So-called conservative exchanges can be carried out in which the amino acid which is replaced has a similar property as the original amino acid, for example, the exchange of Glu by Asp, Gln by Asn, Val by Ile, Leu by Ile, and Ser by Thr. For example, amino acids with similar properties can be Aliphatic amino acids (e.g., Glycine, Alanine, Valine, Leucine, Isoleucine); hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. An amino acid sequence can be modulated with the help of art-known computer simulation programs that can produce a polypeptide with, for example, improved activity or altered regulation. On the basis of these artificially generated polypeptide sequences, a corresponding nucleic acid molecule coding for such a modulated polypeptide can be synthesized in-vitro using the specific codon-usage of the desired host cell.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tₘ) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tₘ=81.5° C.+16.6(log₁₀[Na⁺])+0.41(fraction G/C content)−0.63(% formamide)−(600/l). Furthermore, the Tₘ of a DNA:DNA hybrid is decreased by 1-1.5° C. for every 1% decrease in nucleotide identity (see e.g., Sambrook and Russel, 2006).

Host cells can be transformed using a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transformed cells can be selected and propagated to provide recombinant host cells that comprise the expression vector stably integrated in the host cell genome.

Conservative Substitutions I

| Side Chain Characteristic | Amino Acid |
|---|---|
| Aliphatic Non-polar | G A P I L V |
| Polar-uncharged | C S T M N Q |
| Polar-charged | D E K R |
| Aromatic | H F W Y |
| Other | N Q D E |

Conservative Substitutions II

| Side Chain Characteristic | Amino Acid |
|---|---|
| Non-polar (hydrophobic) | |
| A. Aliphatic: | A L I V P |
| B. Aromatic: | F W |
| C. Sulfur-containing: | M |
| D. Borderline: | G |
| Uncharged-polar | |
| A. Hydroxyl: | S T Y |
| B. Amides: | N Q |
| C. Sulfhydryl: | C |
| D. Borderline: | G |
| Positively Charged | K R H |
| (Basic): | |
| Negatively Charged | D E |
| (Acidic): | |

Conservative Substitutions III

| Original Residue | Exemplary Substitution |
|---|---|
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, |
| | Phe, |
| Leu (L) | Ile, Val, Met, Ala, Phe |
| Lys (K) | Arg, Gln, Asn |
| Met(M) | Leu, Phe, Ile |
| Phe (F) | Leu, Val, Ile, Ala |
| Pro (P) | Gly |
| Ser (S) | Thr |
| Thr (T) | Ser |
| Trp(W) | Tyr, Phe |
| Tyr (Y) | Trp, Phe, Tur, Ser |
| Val (V) | Ile, Leu, Met, Phe, Ala |

Exemplary nucleic acids that may be introduced to a host cell include, for example, DNA sequences or genes from another species, or even genes or sequences which originate with or are present in the same species, but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA that is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Methods of down-regulation or silencing genes are known in the art. For example, expressed protein activity can be down-regulated or eliminated using antisense oligonucleotides (ASOs), protein aptamers, nucleotide aptamers, and RNA interference (RNAi) (e.g., small interfering RNAs (siRNA), short hairpin RNA (shRNA), single guide RNA (sgRNA), and micro RNAs (miRNA) (see e.g., Rinaldi and Wood (2017) Nature Reviews Neurology 14, describing ASO therapies; Fanning and Symonds (2006) Handb Exp Pharmacol. 173, 289-303G, describing hammerhead ribozymes and small hairpin RNA; Helene, et al. (1992) Ann. N.Y. Acad. Sci. 660, 27-36; Maher (1992) Bioassays 14(12): 807-15, describing targeting deoxyribonucleotide sequences; Lee et al. (2006) Curr Opin Chem Biol. 10, 1-8, describing aptamers; Reynolds et al. (2004) Nature Biotechnology 22(3), 326 — 330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinformatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

### Genome Editing

As described herein, a transposable element (TE)-driven isoform of LIN28B can be modulated (e.g., reduced, eliminated) using genome editing.

As described herein, activity, signals, expression, or function can be modulated (e.g., reduced, eliminated) using genome editing (e.g., downregulate, underexpress, knock out, knockdown).

Processes for genome editing are well known; see e.g., Aldi 2018 Nature Communications 9(1911). Except as otherwise noted herein, therefore, the process of the present disclosure can be carried out in accordance with such processes.

For example, genome editing can comprise CRISPR/Cas9, CRISPR-Cpf1, TALEN, or ZNFs. Adequate blockage of transposable element (TE)-driven isoform of LIN28B by genome editing can result in protection from cancer.

As an example, clustered regularly interspaced short palindromic repeats (CRISPR)/CRISPR-associated (Cas) systems are a new class of genome-editing tools that target desired genomic sites in mammalian cells. Recently published type II CRISPR/Cas systems use Cas9 nuclease that is targeted to a genomic site by complexing with a synthetic guide RNA that hybridizes to a 20-nucleotide DNA sequence and immediately preceding an NGG motif recognized by Cas9 (thus, a (N)₂₀NGG target DNA sequence). This results in a double-strand break three nucleotides upstream of the NGG motif. The double strand break instigates either non-homologous end-joining, which is error-prone and conducive to frameshift mutations that knock out gene alleles, or homology-directed repair, which can be exploited with the use of an exogenously introduced double-strand or single-strand DNA repair template to knock in or correct a mutation in the genome. Thus, genomic editing, for example, using CRISPR/Cas systems could be useful tools for therapeutic applications for cancer to target cells by the removal or addition of LIN28B signals (e.g., activate (e.g., CRISPRa), upregulate, overexpress, downregulate LIN28B).

For example, the methods as described herein can comprise a method for altering a target polynucleotide sequence in a cell comprising contacting the polynucleotide sequence with a clustered regularly interspaced short palindromic repeats-associated (Cas) protein.

### Gene Therapy and Genome Editing

Gene therapies can include inserting a functional gene with a viral vector. Gene therapies for cancer are rapidly advancing.

There has recently been an improved landscape for gene therapies. For example, in the first quarter of 2019, there were 372 ongoing gene therapy clinical trials (Alliance for Regenerative Medicine, May 9, 2019).

Any vector known in the art can be used. For example, the vector can be a viral vector selected from retrovirus, lentivirus, herpes, adenovirus, adeno-associated virus (AAV), rabies, Ebola, lentivirus, or hybrids thereof.

### Gene Therapy Strategies.

| | Strategy |
|---|---|
| Viral Vectors | |
| Retroviruses | Retroviruses are RNA viruses transcribing their single- |
| | stranded genome into a double-stranded DNA copy, which |
| | can integrate into host chromosome |
| Adenoviruses (Ad) | Ad can transfect a variety of quiescent and proliferating |
| | cell types from various species and can mediate robust gene |
| | expression |
| Adeno-associated | Recombinant AAV vectors contain no viral DNA and can |
| Viruses (AAV) | carry~4.7 kb of foreign transgenic material. They are |
| | replication defective and can replicate only while coinfecting |
| | with a helper virus |
| Non-viral vectors | |
| plasmid DNA | pDNA has many desired characteristics as a gene therapy |
| (pDNA) | vector; there are no limits on the size or genetic constitution |
| | of DNA, it is relatively inexpensive to supply, and unlike |
| | viruses, antibodies are not generated against DNA in normal |
| | individuals |
| RNAi | RNAi is a powerful tool for gene specific silencing that could |
| | be useful as an enzyme reduction therapy or means to |
| | promote read-through of a premature stop codon |

Gene therapy can allow for the constant delivery of the enzyme directly to target organs and eliminates the need for weekly infusions. Also, correction of a few cells could lead to the enzyme being secreted into the circulation and taken up by their neighboring cells (cross-correction), resulting in widespread correction of the biochemical defects. As such, the number of cells that must be modified with a gene transfer vector is relatively low.

Genetic modification can be performed either ex vivo or in vivo. The ex vivo strategy is based on the modification of cells in culture and transplantation of the modified cell into a patient. Cells that are most commonly considered therapeutic targets for monogenic diseases are stem cells. Advances in the collection and isolation of these cells from a variety of sources have promoted autologous gene therapy as a viable option.

The use of endonucleases for targeted genome editing can solve the limitations presented by the usual gene therapy protocols. These enzymes are custom molecular scissors, allowing cutting DNA into well-defined, perfectly specified pieces, in virtually all cell types. Moreover, they can be delivered to the cells by plasmids that transiently express the nucleases, or by transcribed RNA, avoiding the use of viruses.

### Formulation

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc., may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutically active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

Controlled-release (or sustained-release) preparations may be formulated to extend the activity of the agent(s) and reduce dosage frequency. Controlled-release preparations can also be used to affect the time of onset of action or other characteristics, such as blood levels of the agent, and consequently, affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

### Cancer

Methods and compositions as described herein can be used for the treatment of cancer. As described herein, the AluJb transposable element drives a tumor-specific isoform of LIN28B in several cancer types, including liver cancer, bladder cancer, urothelial cancer, breast cancer, lung cancer, ovarian cancer, melanoma, or stomach cancer. The AluJb-derived isoform constitutes >90% of LIN28B expression in cancers where this tumor-specific isoform is expressed and is associated with worse outcomes. Thus, the compositions and methods of the present disclosure may be particularly suitable for therapeutic use in subjects having these types of cancers.

Other cancers that could be potentially targeted include, for example, can be Acute Lymphoblastic Leukemia (ALL); Acute Myeloid Leukemia (AML); Adrenocortical Carcinoma; AIDS-Related Cancers; Kaposi Sarcoma (Soft Tissue Sarcoma); AIDS-Related Lymphoma (Lymphoma); Primary CNS Lymphoma (Lymphoma); Anal Cancer; Appendix Cancer; Gastrointestinal Carcinoid Tumors; Astrocytomas; Atypical Teratoid/Rhabdoid Tumor, Childhood, Central Nervous System (Brain Cancer); Basal Cell Carcinoma of the Skin; Bile Duct Cancer; Bone Cancer (including Ewing Sarcoma and Osteosarcoma and Malignant Fibrous Histiocytoma); Brain Tumors; Bronchial Tumors; Burkitt Lymphoma; Carcinoid Tumor (Gastrointestinal); Childhood Carcinoid Tumors; Cardiac (Heart) Tumors; Central Nervous System cancer; Atypical Teratoid/Rhabdoid Tumor, Childhood (Brain Cancer); Embryonal Tumors, Childhood (Brain Cancer); Germ Cell Tumor, Childhood (Brain Cancer); Primary CNS Lymphoma; Cervical Cancer; Cholangiocarcinoma; Bile Duct Cancer Chordoma; Chronic Lymphocytic Leukemia (CLL); Chronic Myelogenous Leukemia (CML); Chronic Myeloproliferative Neoplasms; Colorectal Cancer; Craniopharyngioma (Brain Cancer); Cutaneous T-Cell; Ductal Carcinoma In Situ (DCIS); Embryonal Tumors, Central Nervous System, Childhood (Brain Cancer); Endometrial Cancer (Uterine Cancer); Ependymoma, Childhood (Brain Cancer); Esophageal Cancer; Esthesioneuroblastoma; Ewing Sarcoma (Bone Cancer); Extracranial Germ Cell Tumor; Extragonadal Germ Cell Tumor; Eye Cancer; Retinoblastoma; Fallopian Tube Cancer; Fibrous Histiocytoma of Bone, Malignant, or Osteosarcoma; Gallbladder Cancer; Gastric (Stomach) Cancer; Gastrointestinal Carcinoid Tumor; Gastrointestinal Stromal Tumors (GIST) (Soft Tissue Sarcoma); Germ Cell Tumors; Central Nervous System Germ Cell Tumors (Brain Cancer); Childhood Extracranial Germ Cell Tumors; Extragonadal Germ Cell Tumors; Ovarian Germ Cell Tumors; Testicular Cancer; Gestational Trophoblastic Disease; Hairy Cell Leukemia; Head and Neck Cancer; Heart Tumors; Hepatocellular (Liver) Cancer; Histiocytosis, Langerhans Cell; Hodgkin Lymphoma; Hypopharyngeal Cancer; Islet Cell Tumors; Pancreatic Neuroendocrine Tumors; Kaposi Sarcoma (Soft Tissue Sarcoma); Kidney (Renal Cell) Cancer; Langerhans Cell Histiocytosis; Laryngeal Cancer; Leukemia; Lip and Oral Cavity Cancer; Lymphoma; Malignant Fibrous Histiocytoma of Bone or Osteosarcoma; Merkel Cell Carcinoma (Skin Cancer); Mesothelioma, Malignant; Metastatic Cancer; Metastatic Squamous Neck Cancer with Occult Primary; Midline Tract Carcinoma Involving NUT Gene; Mouth Cancer; Multiple Endocrine Neoplasia Syndromes; Multiple Myeloma/Plasma Cell Neoplasms; Mycosis Fungoides (Lymphoma); Myelodysplastic Syndromes, Myelodysplastic/Myeloproliferative Neoplasms; Myelogenous Leukemia, Chronic (CML); Myeloid Leukemia, Acute (AML); Myeloproliferative Neoplasms; Nasal Cavity and Paranasal Sinus Cancer; Nasopharyngeal Cancer; Neuroblastoma; Non-Hodgkin Lymphoma; Non-Small Cell Lung Cancer; Oral Cancer, Lip or Oral Cavity Cancer; Oropharyngeal Cancer; Osteosarcoma and Malignant Fibrous Histiocytoma of Bone; Pancreatic Cancer; Pancreatic Neuroendocrine Tumors (Islet Cell Tumors); Papillomatosis; Paraganglioma; Paranasal Sinus and Nasal Cavity Cancer; Parathyroid Cancer; Penile Cancer; Pharyngeal Cancer; Pheochromocytoma; Pituitary Tumor; Plasma Cell Neoplasm/Multiple Myeloma; Pleuropulmonary Blastoma; Pregnancy and Breast Cancer; Primary Central Nervous System (CNS) Lymphoma; Primary Peritoneal Cancer; Prostate Cancer; Rectal Cancer; Recurrent Cancer Renal Cell (Kidney) Cancer; Retinoblastoma; Rhabdomyosarcoma, Childhood (Soft Tissue Sarcoma); Salivary Gland Cancer; Sarcoma; Childhood Rhabdomyosarcoma (Soft Tissue Sarcoma); Childhood Vascular Tumors (Soft Tissue Sarcoma); Ewing Sarcoma (Bone Cancer); Kaposi Sarcoma (Soft Tissue Sarcoma); Osteosarcoma (Bone Cancer); Uterine Sarcoma; Sèzary Syndrome (Lymphoma); Skin Cancer; Small Cell Lung Cancer; Small Intestine Cancer; Soft Tissue Sarcoma; Squamous Cell Carcinoma of the Skin; Squamous Neck Cancer with Occult Primary, Metastatic; T-Cell Lymphoma, Cutaneous; Lymphoma; Mycosis Fungoides and Sezary Syndrome; Testicular Cancer; Throat Cancer; Nasopharyngeal Cancer; Oropharyngeal Cancer; Hypopharyngeal Cancer; Thymoma and Thymic Carcinoma; Thyroid Cancer; Thyroid Tumors; Transitional Cell Cancer of the Renal Pelvis and Ureter (Kidney (Renal Cell) Cancer); Ureter and Renal Pelvis; Transitional Cell Cancer (Kidney (Renal Cell) Cancer; Urethral Cancer; Uterine Cancer, Endometrial; Uterine Sarcoma; Vaginal Cancer; Vascular Tumors (Soft Tissue Sarcoma); Vulvar Cancer; or Wilms Tumor.

### Therapeutic Methods

Also provided is a process of treating cancer in a subject in need of administration of a therapeutically effective amount of a splice-switching oligonucleotide (SSO) targeted to a nucleic acid encoding a transposable element (TE)-driven isoform of LIN28B, so as to reduce LIN28B expression or reduce cancer cell viability.

Methods described herein are generally performed on a subject in need thereof. A subject in need of the therapeutic methods described herein can be a subject having, diagnosed with, suspected of having, or at risk for developing cancer. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective amount of an SSO targeted to a nucleic acid encoding a TE-driven isoform of LIN28B is, for example, an amount that would cause the desired therapeutic effect in a subject while minimizing undesired side effects. In various embodiments, an effective amount of an SSO targeted to a nucleic acid encoding a TE-driven isoform of LIN28B is described herein can substantially inhibit LIN28B expression, slow the progress of cancer, or limit the development of cancer.

According to the methods described herein, administration can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic, buccal, or rectal administration.

When used in the treatments described herein, a therapeutically effective amount of an SSO targeted to a nucleic acid encoding a TE-driven isoform of LIN28B can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to reduce cancer cell viability.

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; the activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4^{th} ed., Lippincott Williams & Wilkins, ISBN 0781741475; Shawl (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes reversing, or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or a physician.

Administration of an SSO targeted to a nucleic acid encoding a TE-driven isoform of LIN28B can occur as a single event or over a time course of treatment. For example, an SSO targeted to a nucleic acid encoding a TE-driven isoform of LIN28B can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to or before, concurrent with, or after conventional treatment modalities for cancer.

An SSO targeted to a nucleic acid encoding a TE-driven isoform of LIN28B can be administered simultaneously or sequentially with another agent, such as an antibiotic, an anti-inflammatory, or another agent. For example, SSO targeted to a nucleic acid encoding a TE-driven isoform of LIN28B can be administered simultaneously with another agent, such as an antibiotic or an anti-inflammatory. Simultaneous administration can occur through administration of separate compositions, each containing one or more of SSO targeted to a nucleic acid encoding TE-driven isoform of LIN28B, an antibiotic, an anti-inflammatory, or another agent. Simultaneous administration can occur through administration of one composition containing two or more of an SSO targeted to a nucleic acid encoding a TE-driven isoform of LIN28B, an antibiotic, an anti-inflammatory, or another agent. An SSO targeted to a nucleic acid encoding a TE-driven isoform of LIN28B can be administered sequentially with an antibiotic, an anti-inflammatory, or another agent. For example, an SSO targeted to a nucleic acid encoding a TE-driven isoform of LIN28B can be administered before or after administration of an antibiotic, an anti-inflammatory, or another agent.

Active compounds are administered at a therapeutically effective dosage sufficient to treat a condition associated with a condition in a patient. For example, the efficacy of a compound can be evaluated in an animal model system that may be predictive of efficacy in treating the disease in a human or another animal, such as the model systems shown in the examples and drawings.

An effective dose range of a therapeutic can be extrapolated from effective doses determined in animal studies for a variety of different animals. In general, a human equivalent dose (HED) in mg/kg can be calculated in accordance with the following formula (see e.g., Reagan-Shaw et al., FASEB J., 22(3):659-661, 2008, which is incorporated herein by reference):

HED (mg/kg)=Animal dose (mg/kg)×(Animal Kₘ/Human Kₘ)

Use of the Kₘ factors in conversion results in more accurate HED values, which are based on body surface area (BSA) rather than only on body mass. Kₘ values for humans and various animals are well known. For example, the Kₘ for an average 60 kg human (with a BSA of 1.6 m²) is 37, whereas a 20 kg child (BSA 0.8 m²) would have a Kₘ of 25. Kₘ for some relevant animal models are also well known, including: mice Kₘ of 3 (given a weight of 0.02 kg and BSA of 0.007); hamster Kₘ of 5 (given a weight of 0.08 kg and BSA of 0.02); rat Kₘ of 6 (given a weight of 0.15 kg and BSA of 0.025) and monkey Kₘ of 12 (given a weight of 3 kg and BSA of 0.24).

Precise amounts of the therapeutic composition depend on the judgment of the practitioner and are peculiar to each individual. Nonetheless, a calculated HED dose provides a general guide. Other factors affecting the dose include the physical and clinical state of the patient, the route of administration, the intended goal of treatment, and the potency, stability, and toxicity of the particular therapeutic formulation.

The actual dosage amount of a compound of the present disclosure or composition comprising a compound of the present disclosure administered to a subject may be determined by physical and physiological factors such as type of animal treated, age, sex, body weight, severity of condition, the type of disease being treated, previous or concurrent therapeutic interventions, idiopathy of the subject and on the route of administration. These factors may be determined by a skilled artisan. The practitioner responsible for administration will typically determine the concentration of active ingredient(s) in a composition and appropriate dose(s) for the individual subject. The dosage may be adjusted by the individual physician in the event of any complication.

In some embodiments, SSO targeted to a nucleic acid encoding a TE-driven isoform of LIN28B may be administered in an amount from about 1 mg/kg to about 100 mg/kg, or about 1 mg/kg to about 50 mg/kg, or about 1 mg/kg to about 25 mg/kg, or about 1 mg/kg to about 15 mg/kg, or about 1 mg/kg to about 10 mg/kg, or about 1 mg/kg to about 5 mg/kg, or about 3 mg/kg. In some embodiments, an SSO targeted to a nucleic acid encoding a TE-driven isoform of LIN28B such as an SSO comprising a sequence ATAAACTGACCTGGCGGCGGTG may be administered in a range of about 1 mg/kg to about 200 mg/kg, or about 50 mg/kg to about 200 mg/kg, or about 50 mg/kg to about 100 mg/kg, or about 75 mg/kg to about 100 mg/kg, or about 100 mg/kg.

The effective amount may be less than 1 mg/kg/day, less than 500 mg/kg/day, less than 250 mg/kg/day, less than 100 mg/kg/day, less than 50 mg/kg/day, less than 25 mg/kg/day or less than 10 mg/kg/day. It may alternatively be in the range of 1 mg/kg/day to 200 mg/kg/day.

In other non-limiting examples, a dose may also comprise from about 1 micro-gram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

### Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency; improve taste of the product; or improve shelf life of the product.

### Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to splice-switching oligonucleotides and pharmaceutical carriers. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal, or any other material typically employed to hold reagents. Other examples of suitable containers include bottles that may be fabricated from similar substances as ampules and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or another substrate, and/or may be supplied as an electronic-readable medium or video. Detailed instructions may not be physically associated with the kit; instead, a user may be directed to an Internet web site specified by the manufacturer or distributor of the kit.

A control sample or a reference sample as described herein can be a sample from a healthy subject or sample, a wild-type subject or sample, or from populations thereof. A reference value can be used in place of a control or reference sample, which was previously obtained from a healthy subject or a group of healthy subjects or a wild-type subject or sample. A control sample or a reference sample can also be a sample with a known amount of a detectable compound or a spiked sample.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1: Transposable Element-Directed LIN28B Abrogation Through Splice-Switching Oligonucleotides (TED LASSO)

This Example describes generation and testing of fully 2′-O-methoxyethyl modified SSOs containing phosphorothioate internucleotide linkages, directed against the AluJb transposable element (TE)-driven, cancer-specific isoform of LIN28B.

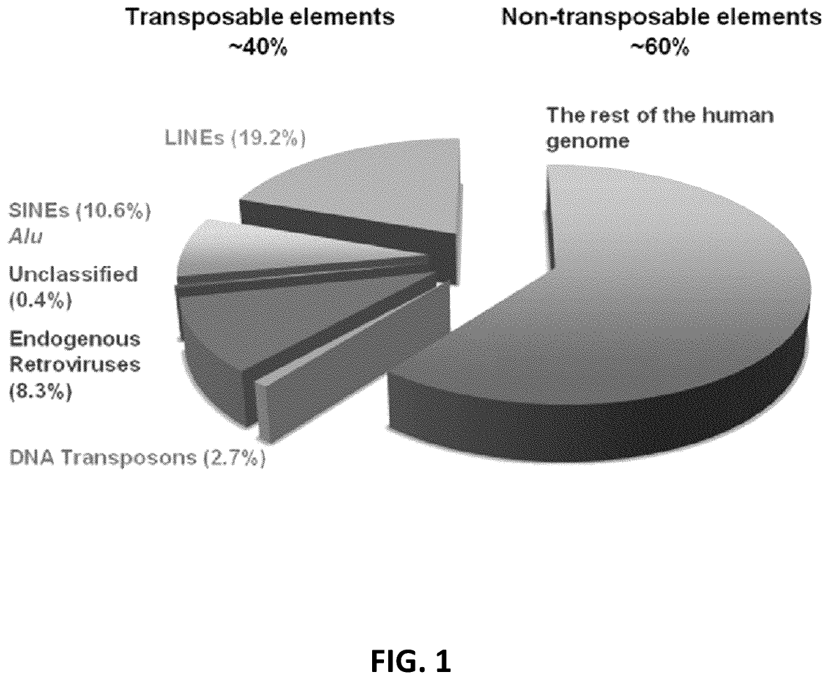

Transposable elements (TEs) constitute significant proportions of genome content (see e.g., FIG. 1). TEs are sequences in the genome that currently have or once have had the ability to mobilize, and they account for nearly 50% of total human genomic content. TEs contribute cryptic promoters genome-wide that may be silenced by epigenetic mechanisms, such as DNA methylation. Their sequences are often repetitive complicating their analysis in genomic studies of pathology. However, in recent years improvements in bioinformatics and genomics technologies have enabled an improved understanding of their roles in disease processes.

TE promoter exaptation, e.g., wherein transcription is initiated at a TE-derived promoter, drives in-frame oncogene expression in cancer. It was previously demonstrated that alternative transcription start sites contributed by TEs throughout the genome can become activated in cancer (see e.g., Jang et al. (2019) Nat Genet. 51(4):611-617). In normal states of cellular physiology, TEs are repressed through epigenetic mechanisms such as DNA methylation, but the hypomethylated epigenome characteristic of cancer supports their reactivation. When transcription is initiated at a TE-derived promoter, transcription can readthrough and splice into downstream gene sequences resulting in chimeric transcripts with concatenated TE and canonical gene sequences.

These transcripts can be translated in-frame of the downstream gene resulting in functional overexpression with TE-derived prepended amino acids or they can be translated out of frame resulting in novel peptide sequences.

In the case of functional overexpression, if the downstream gene is an oncogene, this can lead to demonstrable fitness advantages for the cancer cell (e.g., onco-exaptation). Expression of the oncogene LIN28B has been shown to be driven primarily by the de-repression of the AluJb TE 20kb upstream of the canonical LIN28B promoter and that CRISPR knockout of this promoter resulted in reduced growth, migration and tumorigenicity of cells expressing this onco-exaptation transcript (see e.g., Jang et al. (2019) Nat Genet. 51(4):611-617). Different omics methods including CAGE-seq, WGBS and ATAC-seq were used to demonstrate the cancer-associated expression of the TE-derived isoform of LIN28B in the lung adenocarcinoma cell line H1299, whereas transcription is essentially undetectable and epigenetically impermissive at the canonical LIN28B promoter. Further, it was shown that AluJb-LIN28B drives worse hepatocellular carcinoma outcomes in patients and that AluJb-LIN28B contributes to H1299 tumorigenicity.

RNA splicing depends on splice factor binding of transcript. Steric hindrance of spliceosome function alters the reading frame, producing a premature stop codon. Knockdown occurs through nonsense-mediated decay. For example, nusinersen, a fully phosphorothioate (PS)/2′-O-methoxyethyl (2′-MOE) antisense oligonucleotide is FDA approved for the treatment of spinal muscular atrophy (SMA).

As demonstrated herein, this rationale was applied to the AluJb-LIN28b onco-exaptation event. LIN28B is a potent oncogene involved in ˜10% of more phenotypically aggressive liver cancers. In these cancers nearly all of the total LIN28B gene expression is driven by promoter de-repression of the AluJb TE ˜20 kb upstream. As this particular incident of alternative promoter usage was demonstrated to be highly tumor-specific, it makes a compelling target for a precision therapy.

The first task was to figure out which sequences would be best to block the tumor-specific LIN28B isoform that would not affect canonical LIN28B wherever it might be expressed physiologically. The exon 1-intron 1 junction (SEQ ID NO: 3) and intron 1-exon 2 junction (SEQ ID NO: 4) of AluJb-LIN28B were selected as the “search space” for tumor-specific sequences targetable by SSOs; the second exon splices into the second exon of the canonical isoform of LIN28B, which is not tumor-specific.

The tumor-specific exons in the AluJb-LIN28b oncogene were entered into ESEfinder and spliceAid—two open-source motif prediction tools—and the most desirable SSO sequences were identified as those that overlap with predicted splice factor binding sites and potentially interfere with spliceosomal function if sterically blocked while having little homology to other expressed off-target genes. The off-target analysis was performed by BLAST search.

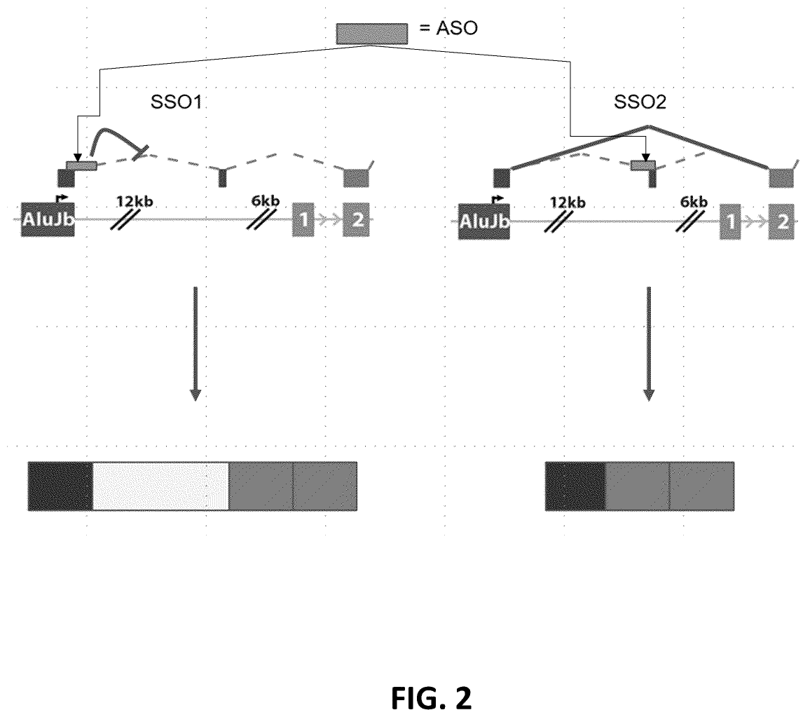

The analysis identified antisense oligonucleotides SSO1 (SEQ ID NO: 1) and SSO2 (SEQ ID NO: 2) fulfilling the above criterial. SSO1 and SSO2 were synthesized and ordered from IDT along with their associated sequence scrambled negative controls, SCR1 (SEQ ID NO: 5) and SCR2 (SEQ ID NO: 6). Their schematized mechanisms of action, intron 1 retention for SSO1 and exon 2 skipping for SSO2, are depicted in FIG. 2.

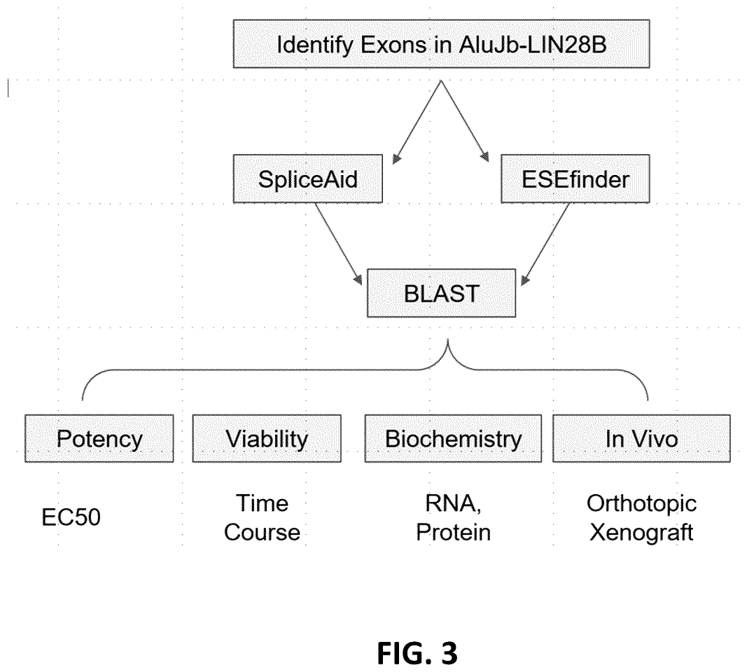

Next, SSO1 and SSO2 were evaluated in AluJb-LIN28B-expressing and canonical LIN28B-expressing cell lines, H1299 and HEK293, respectively (see e.g., FIG. 3).

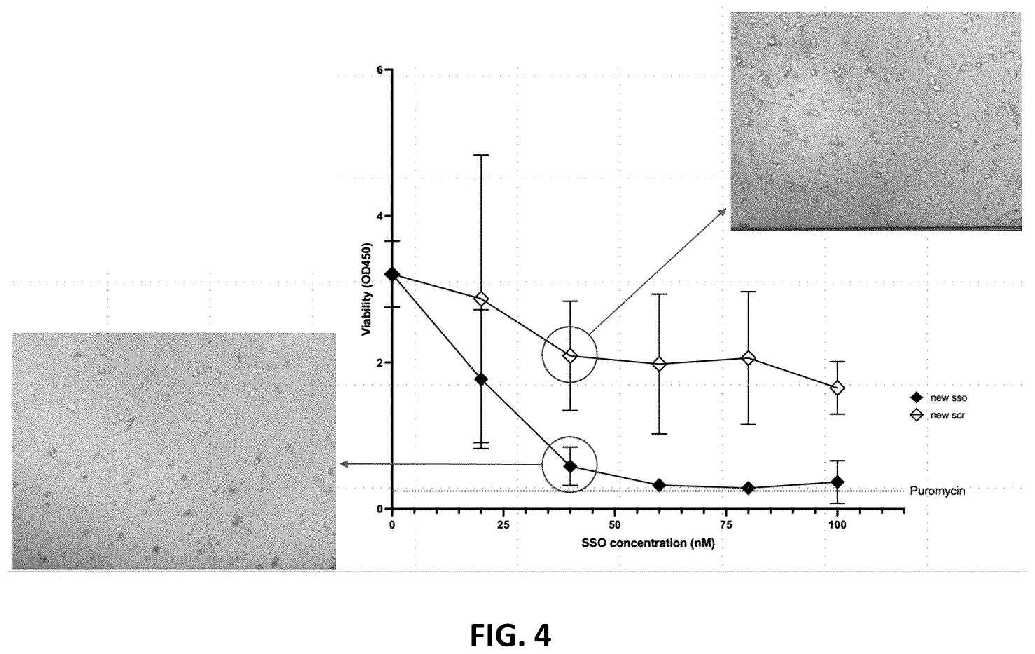

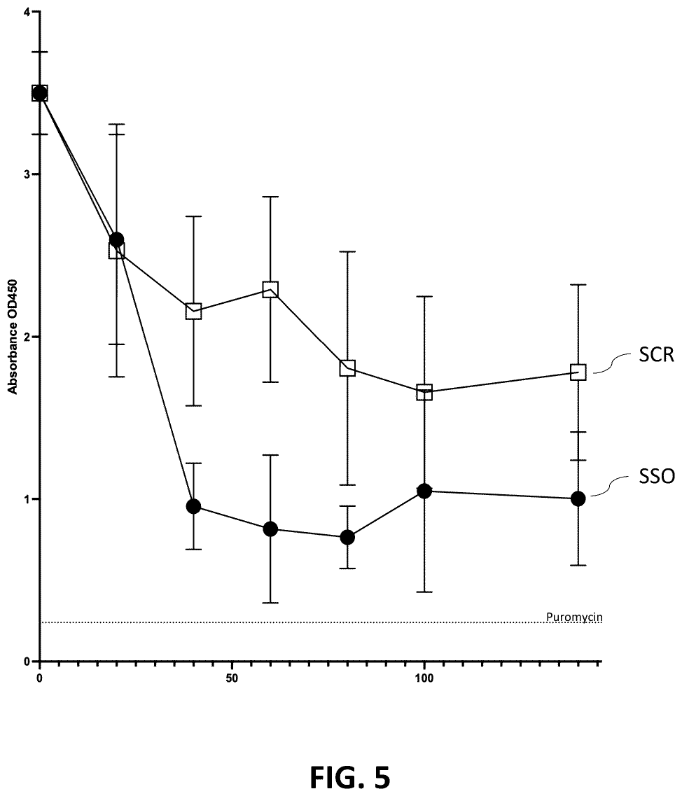

First, viability of H1299 cells (cancer cells that express AluJb-LIN28B) was quantified after five days of exposure to SSO1 at 0-100 nM compared to controls (see e.g., FIG. 4). A significant viability difference emerged at concentrations as low as 40 nM, and the cells were as confluent as those treated for 5 days at 100 nM, suggesting a saturating effect on cell viability. Similarly, SSO2 was also found to reduce H1299 cell viability (see e.g., FIG. 5).

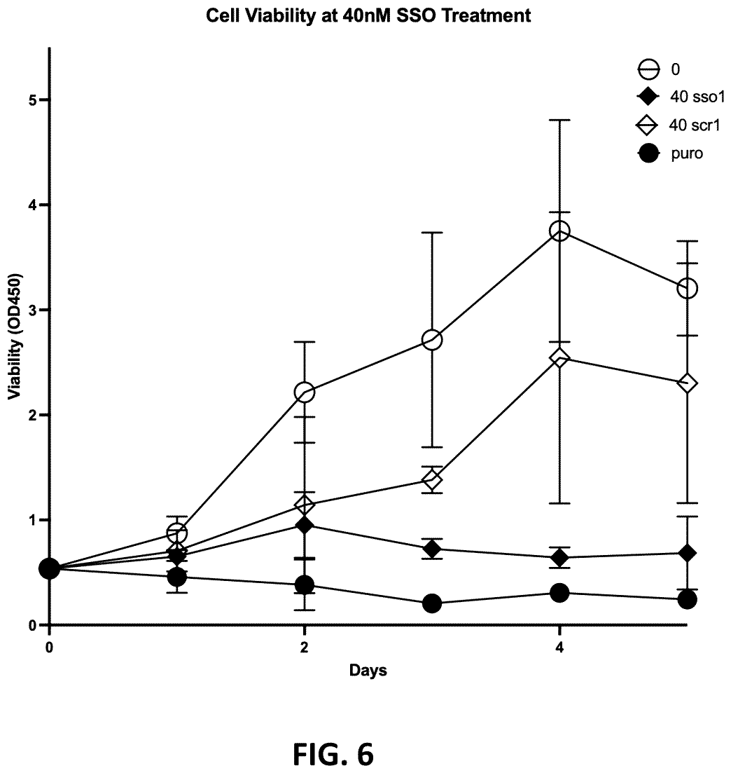

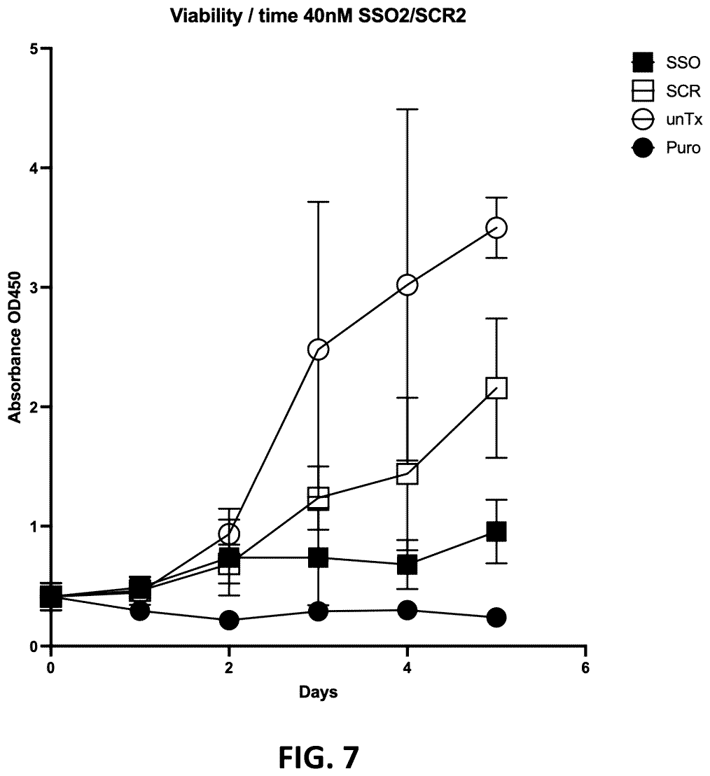

Next, it was investigated as to what time course the viability difference occurs for cells treated with SSO1 or SSO2 as compared to controls. At 40 nM SSO1, a significant viability difference emerged at day 3 after transfection and was durably maintained over the next 2 days for the SSO1 treated cells, but not for the scrambled negative control (see e.g., FIG. 6). For SSO2 treated cells, a significant viability differenced emerged at day 5 after transfection (see e.g., FIG. 7).

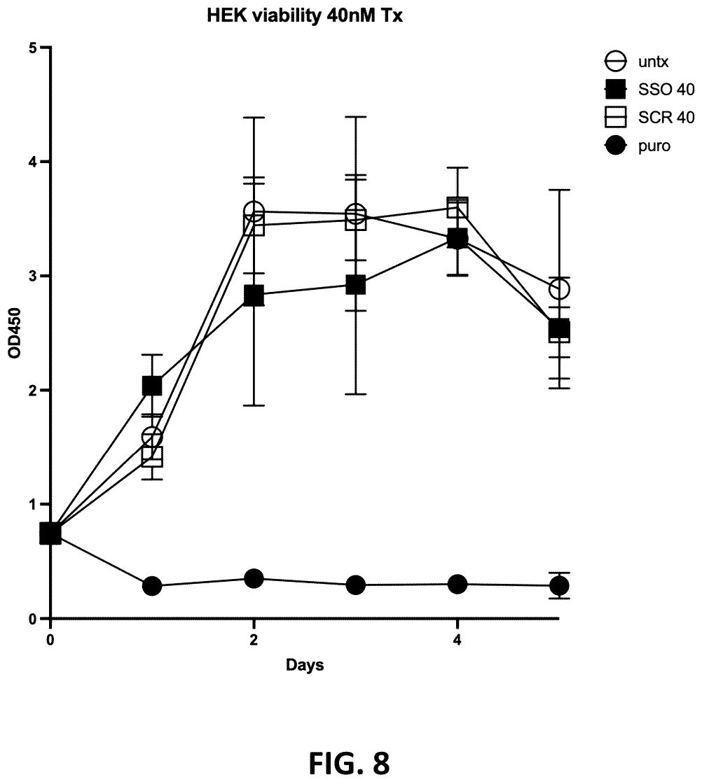

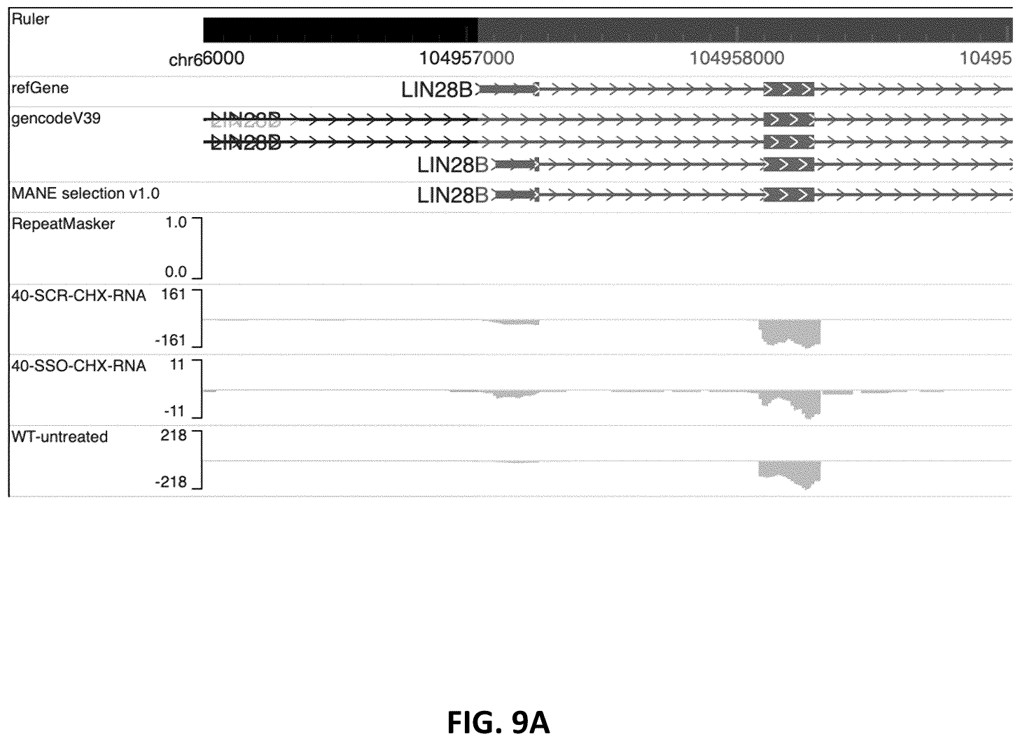

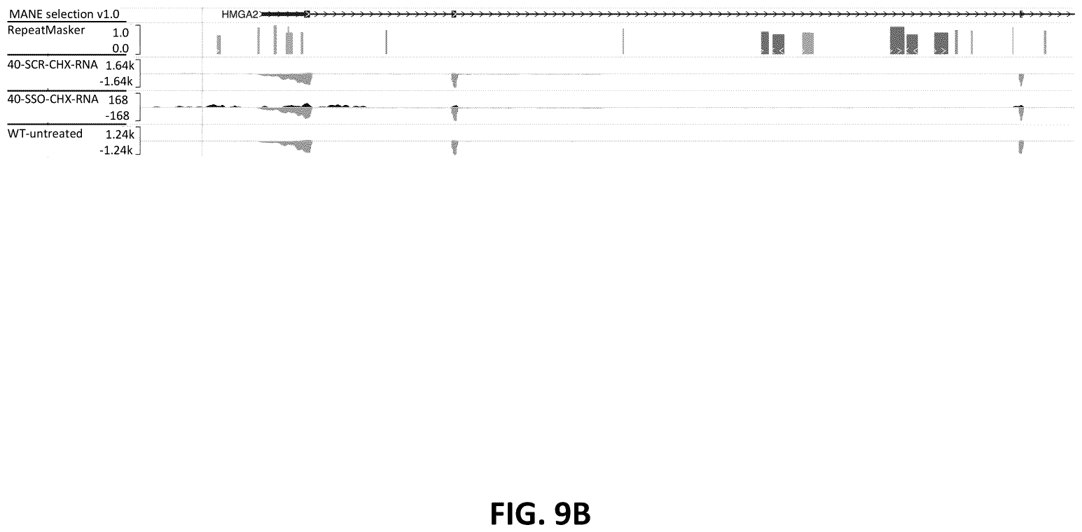

The effect on viability observed with SSO1 transfection was then assessed in HEK293 cells, which express only LIN28B driven by its canonical promoter (see e.g., FIG. 8). No difference at any time point emerged in any of the treatment groups, except the positive control puromycin. Knockdown of LIN28B was confirmed by RNA-seq (see e.g., FIG. 9A). HMGA2 expression was also measured (see e.g., FIG. 9B), as HMGA2 is a let-7 regulated oncogene and LIN28B represses let-7 miRNA.

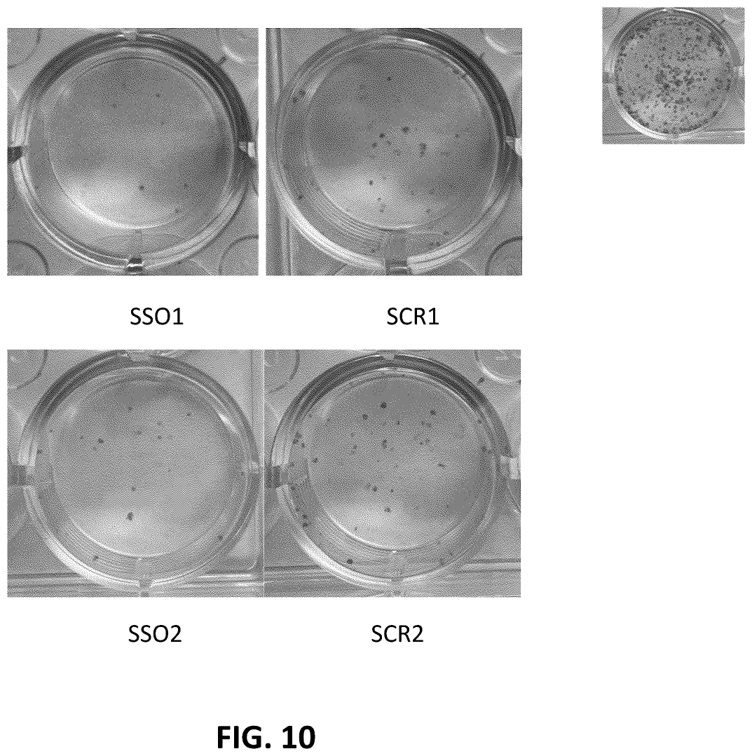

SSO1 and SSO2 treatment also reduced H1299 colony formation ability (see e.g., FIG. 10). A colony formation assay or clonogenic assay tests cells for ability to undergo “unlimited division” and can be used to discern cytotoxic vs. cytostatic effect of the SSOs.

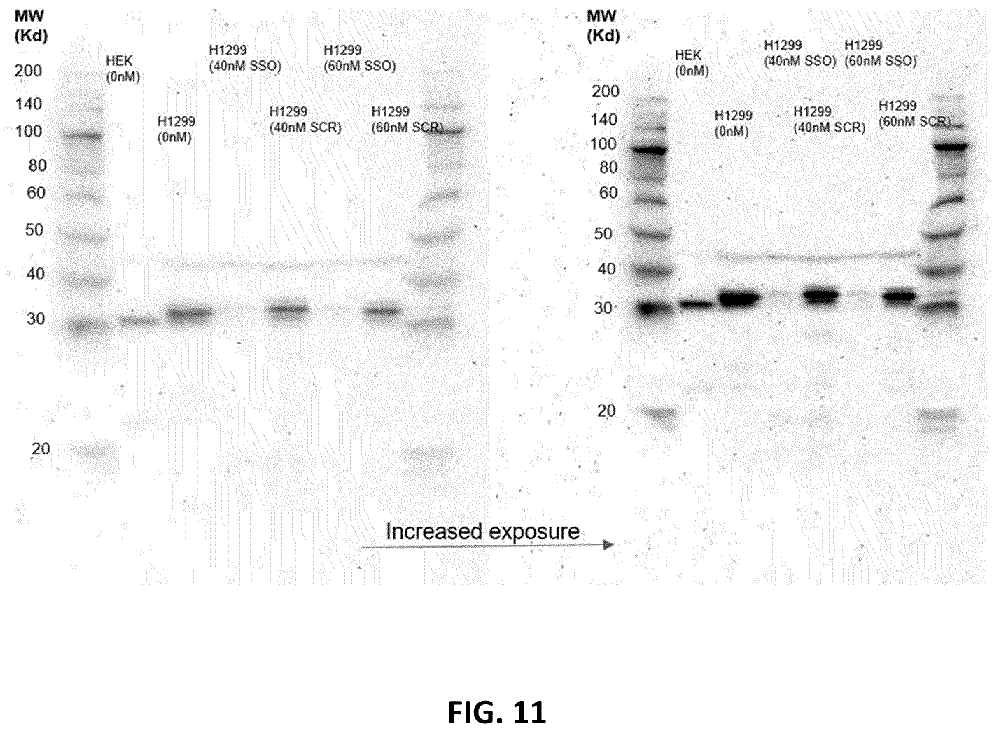

Western blot confirmed that SSO1 reduced in-frame LIN28B protein expression in H1299 (see e.g., FIG. 11). No further reduction was observed at 60 nM vs 40 nm SSO1, consistent with the EC50 data predicting a maximal effective concentration of 40 nM (see e.g., FIG. 4).

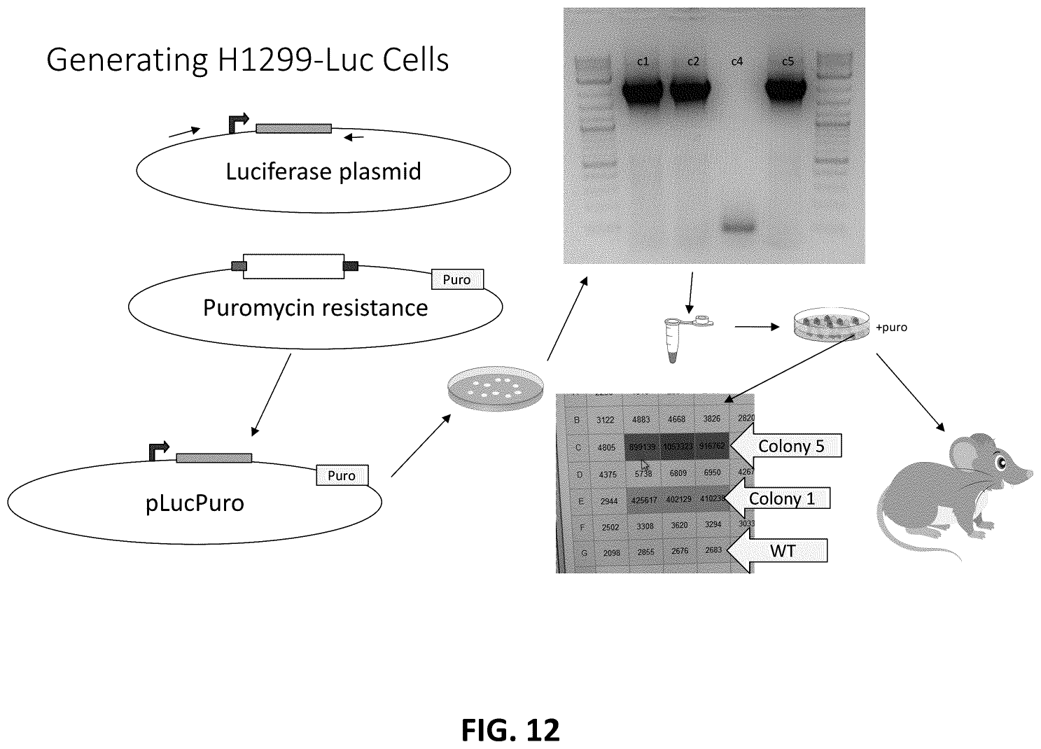

The effect of SSO1 and SSO2 is tested in vivo using an athymic nude mouse host according to previously described methods (see e.g., Justilien and Fields (2013) Curr Protoc Pharmacol. 8:62:14.27.1-14.27.17). An H1299-Luc orthotopic xenograft (see e.g., FIG. 12) will be delivered by intercostal injection. IVIS50 imaging will be used to track tumor growth after treatment with SSO1 or SSO2 compared to scrambled controls or vehicle. Twice weekly body weight, IV injection of SSO in saline, and bioluminescent image will be used. 30 mice total will be used, with 5 per treatment group and 5 for implantation protocol optimization.

What is claimed is:

1. A method of treating cancer in a subject in need thereof, the method comprising administering to the subject a splice-switching oligonucleotide (SSO) targeted to a nucleic acid encoding a transposable element (TE)-driven isoform of LIN28B.

2. The method of claim 1, wherein the SSO is targeted to an exon-intron junction or an intron-exon junction of the TE-driven isoform of LIN28B.

3. The method of claim 1, wherein the TE-driven isoform of LIN28B is AluJb-LIN28B.

4. The method of claim 1, wherein the SSO is at least 50% identical to SEQ ID NO: 1 or SEQ ID NO: 2, or to a corresponding reverse, complement, or reverse-complement sequence thereof.

5. The method of claim 1, wherein the SSO is at least 50% identical to SEQ ID NO: 3 or SEQ ID NO: 4, or to a corresponding reverse, complement, or reverse-complement sequence thereof and is at least 10 nucleotides in length.

6. The method of claim 1, wherein the SSO comprises SEQ ID NO: 1, SEQ ID NO: 2, or a corresponding reverse, complement, or reverse-complement sequence thereof.

7. The method of claim 1, wherein the subject has a cancer that expresses AluJb-LIN28B.

8. The method of claim 7, wherein the cancer is selected from liver cancer, bladder cancer, urothelial cancer, breast cancer, lung cancer, ovarian cancer, melanoma, and stomach cancer.

9. The method of claim 1, wherein the SSO comprises at least one chemical modification.

10. The method of claim 9, wherein the chemical modification is selected from the group consisting of 2′-O-methoxyethyl modification, phosphorothioate internucleotide linkage, and combinations thereof.

11. The method of claim 1, wherein the nucleic acid encoding the TE-driven isoform of LIN28B is a pre-mRNA.

12. The method of claim 1, wherein administering the SSO reduces at least one of a mRNA level of the TE-driven isoform of LIN28B, a protein level of the TE-driven isoform of LIN28B, and cancer cell viability in the subject.

13. A pharmaceutical composition comprising a splice-switching oligonucleotide (SSO) targeted to a nucleic acid encoding a transposable-element (TE)-driven isoform of LIN28B and a pharmaceutically acceptable carrier.

14. The pharmaceutical composition of claim 13, wherein the SSO is targeted to an exon-intron junction of the TE-driven isoform of LIN28B.

15. The pharmaceutical composition of claim 13, wherein the TE-driven isoform of LIN28B is AluJb-LIN28B.

16. The pharmaceutical composition of claim 13, wherein the SSO is at least 50% identical to SEQ ID NO: 1 or SEQ ID NO: 2, or to a corresponding reverse, complement, or reverse-complement sequence thereof.

17. The pharmaceutical composition of claim 13, wherein the SSO is at least 50% identical to SEQ ID NO: 3 or SEQ ID NO: 4, or to a corresponding, reverse, complement, or reverse-complement sequence thereof, and is at least 10 nucleotides in length.

18. The pharmaceutical composition of claim 13, wherein the SSO comprises SEQ ID NO: 1, SEQ ID NO: 2, or a corresponding reverse, complement, or reverse-complement sequence thereof.

19. The pharmaceutical composition of claim 13, wherein the SSO comprises at least one chemical modification.

20. The composition of claim 19, wherein the chemical modification is selected from the group consisting of 2′-O-methoxyethyl modification, phosphorothioate internucleotide linkage, and combinations thereof.
